(19) United States
(12) Patent Application Publication
(10) Pub. No.: US 2021/0074390 A1
(43) Pub. Date: Mar. 11, 2021

(54) Urinalysis Devices and Related Methods

(71) Applicant: Fresenius Medical Care Holdings, Inc., Waltham, MA (US)

(72) Inventors: Sameera Anirudh Peesapati, Pittsburg, CA (US); David Yuds, Hudson, NH (US)

(21) Appl. No.: 16/567,265

(22) Filed: Sep. 11, 2019

(51) Int. Cl. G16H 10/40; G16H 10/60; G06K 7/10; G01N 31/22; G01N 33/493

(52) U.S. Cl. CPC G16H 10/40; G16H 10/60; G01N 33/493; G01N 31/221; G06K 7/10297

(57) ABSTRACT

This disclosure relates to method that comprises sensing properties of a urine sample of a patient using a biomarker sensor to generate raw urine data. The method further includes transmitting the raw urine data to a processor, using the processor to generate processed urine data from the raw urine data, and transmitting the processed urine data to a computing device.

### TECHNICAL FIELD

This disclosure relates to urinalysis devices and related methods.

### BACKGROUND

Dialysis is a treatment used to support a patient with insufficient renal function. The two principal dialysis methods are hemodialysis and peritoneal dialysis.

During hemodialysis (“HD”), the patient's blood is passed through a dialyzer of a dialysis machine while also passing a dialysis solution or dialysate through the dialyzer. A semi-permeable membrane in the dialyzer separates the blood from the dialysate within the dialyzer and allows diffusion and osmosis exchanges to take place between the dialysate and the blood stream. These exchanges across the membrane result in the removal of waste products, including solutes like urea and creatinine, from the blood. These exchanges also regulate the levels of other substances, such as sodium and water, in the blood. In this way, the dialysis machine acts as an artificial kidney for cleansing the blood.

During peritoneal dialysis (“PD”), a patient's peritoneal cavity is periodically infused with dialysis solution or dialysate. The membranous lining of the patient's peritoneum acts as a natural semi-permeable membrane that allows diffusion and osmosis exchanges to take place between the solution and the blood stream. These exchanges across the patient's peritoneum, like the continuous exchange across the dialyzer in HD, result in the removal of waste products, including solutes like urea and creatinine, from the blood, and regulate the levels of other substances, such as sodium and water, in the blood.

Many PD machines are designed to automatically infuse, dwell, and drain dialysate to and from the patient's peritoneal cavity. The treatment typically lasts for several hours, often beginning with an initial drain cycle to empty the peritoneal cavity of used or spent dialysate. The sequence then proceeds through the succession of fill, dwell, and drain phases that follow one after the other. Each phase is called a cycle.

### SUMMARY

In certain aspects, a method includes sensing properties of a urine sample of a patient using a biomarker sensor to generate raw urine data, transmitting the raw urine data to a processor, using the processor to generate processed urine data from the raw urine data, and transmitting the processed urine data to a computing device.

In some embodiments, the method further includes suggesting one or more adjustments to a treatment plan based on the processed urine data.

In some embodiments, the treatment plan is at least one of a nutrition plan, a medication prescription, and a blood treatment prescription.

In some embodiments, the processed urine data includes a packet of urine data that contains levels and concentrations of the sensed properties in the urine sample.

In some embodiments, the method further includes sensing a patient identification, generating patient identification data, and transmitting the patient identification data to the processor.

In some embodiments, the method further includes comparing the transmitted patient identification data to predetermined patient identification data and determining a patient identification.

In some embodiments, the method further includes linking the processed urine data to the patient identification.

In some embodiments, the method further includes linking the transmitted raw urine data to the transmitted patient identification data.

In some embodiments, sensing the patient identification includes sensing a fingerprint using a fingerprint reader.

In some embodiments, sensing the patient identification includes sensing an RFID tag using an RFID detector.

In some embodiments, transmitting the raw urine data to the processor includes transmitting the raw urine data using a signal transmitter of a urinalysis device, receiving the raw urine data using a signal receiver of an intermediate device, and transmitting the raw urine data to the processor using a signal transmitter of the intermediate device.

In some embodiments, the intermediate device is a mobile computing device.

In some embodiments, the mobile computing device is a mobile phone or a tablet.

In some embodiments, the method further includes using the processor to adjust parameters of a blood treatment prescription based on the processed urine data.

In some embodiments, the method further includes transmitting the blood treatment prescription with the adjusted parameters to a blood treatment machine.

In some embodiments, the processed urine data includes at least one of specific gravity, pH, uric acid levels, glucose levels, fat levels, proteins levels, fiber levels, nitrite levels, sodium levels, potassium levels, calcium levels, and phosphate levels.

In some embodiments, the method further includes generating a report based on the processed urine data.

In some embodiments, the report identifies properties of the urine sample that are outside a predetermined range.

In some embodiments, the method further includes determining a diet of a patient based on the processed urine data, wherein the report identifies the diet of the patient.

In some embodiments, the method further includes transmitting the report to a medical professional.

In some embodiments, the method further includes transmitting the report to a patient.

In certain aspects, a urinalysis device includes a body configured to be connected to a toilet, a biomarker sensor attached to a portion of the body configured to be submerged in water in the toilet when the body is connected to the toilet, the biomarker sensor configured to detect properties of urine and to generate raw urine data including the detected properties of the urine, and a signal transmitter configured to transmit the raw urine data to a processing device.

In some embodiments, the urinalysis device further includes a patient identification detector.

In some embodiments, the patient identification detector is a fingerprint reader.

In some embodiments, the patient identification detector is an RFID detector.

In some embodiments, the patient identification detector is configured to generate patient identification data, and the signal transmitter is configured to transmit the patient identification data to the processing device.

In some embodiments, the body includes a first arm, a second arm, and a bar connecting the first arm to the second arm, wherein the first arm, the second arm, and the bar cooperate to define a cavity configured to receive a rim of the toilet, and the biomarker sensor is disposed on the first arm.

In some embodiments, the urinalysis device further includes an LED indicator configured to indicate a state of the urinalysis device, and a battery configured to power the biomarker sensor, the signal transmitter, and the LED.

In some embodiments, the processing device is a remote server.

In certain aspects, a system includes a urinalysis device and a processing device. The urinalysis device includes a body configured to be connected to a toilet, a biomarker sensor attached to a portion of the body configured to be submerged in water in the toilet when the body is connected to the toilet, the biomarker sensor configured to detect properties of urine and to generate raw urine data including the detected properties of the urine, and a signal transmitter configured to transmit the raw urine data. The processing device includes a signal receiver configured to receive the raw urine data from the signal transmitter of the urinalysis device, a processor configured to process the raw urine data and generate processed urine data from the raw urine data, and a memory for storing the processed urine data.

In some embodiments, the urinalysis device is in wireless communication with the processing device.

In some embodiments, the processing device is a mobile phone or tablet.

In some embodiments, the mobile phone or tablet is wirelessly connected to a blood treatment machine and is configured to transmit the processed urine data to the blood treatment machine.

In some embodiments, the processing device is a blood treatment machine.

In some embodiments, the blood treatment machine includes a memory and a controller configured to adjust saved blood treatment parameters based on the processed urine data.

In some embodiments, the processing device is a computer.

In some embodiments, the computer is wirelessly connected to a blood treatment machine and is configured to transmit the processed urine data to the blood treatment machine.

Embodiments can include one or more of the following advantages.

The urinalysis device and associated methods may improve patient care by providing data related to physiological parameters relevant to renal function in the patient to appropriate medical staff, such as a nutritionist, clinician, or doctor. The data can, for example, be used by the nutritionist, clinician, and/or doctor to determine a suitable diet for the patient, promote patient adherence to the diet, determine appropriate medications for the patient, and/or determine appropriate prescriptions to be used for blood treatments.

The device may also improve patient care by tracking and saving detailed records of patient data in a server for later use and analysis. The data may, for example, be used to show trends and to figure out what combination of diet, medications, and treatment are most effective for the patient.

The urinalysis device can be used to accurately monitor a patient's diet without relying on the patient to manually log diet information. This makes tracking dietary information much easier for the patient, as compared to a manually logging process. This can also decrease the chance of user and/or input error.

In some cases, the server is connected to both the urinalysis device and a blood treatment machine so that data received by the server from the urinalysis device can be processed and then transmitted to the blood treatment machine. This can allow the clinician to access the processed data at the blood treatment machine. In some cases, the server can process the data received from the urinalysis device to generate one or more recommended blood treatment prescriptions, which can be transmitted to the blood treatment machine. The server can, for example, determine a combination of blood treatment parameters, such as the concentration of the dialysate to be used, the target ultrafiltration volume, the blood flow rate, the dialysate flow rate, etc., to be included in the prescription(s). The clinician can then simply select the prescription to be used for the blood treatment without having to manually enter the various blood treatment parameters. This can save the clinician a lot of time and can result in a more optimized blood treatment for the patient.

In addition to transmitting processed data to a patient's medical staff and/or the blood treatment machine, the processed data can be transmitted to the patient (e.g., to a patient's electronic device, such as a mobile phone or tablet). This can advantageously allow the patient to more closely monitor the impact of his or her treatment plan, including his or her diet, medication regimen, and blood treatments.

The details of one or more embodiments of the invention are set forth in the accompanying drawings and the description below. Other aspects, features, and advantages will be apparent from the description and drawings, and from the claims.

### BRIEF DESCRIPTION OF FIGURES

FIG. 1 illustrates a system including a urinalysis device that is releasably attached to a toilet and in electronic communication with a server, which is in communication with patient and clinician devices.

FIG. 2 illustrates the urinalysis device of FIG. 1.

FIGS. 3A and 3B are schematic illustrations of a biomarker sensor used in the urinalysis device of FIG. 1.

FIG. 4 is a flowchart showing a method of using the system of FIG. 1.

FIG. 5 illustrates an alternative cup-shaped urinalysis device.

### DETAILED DESCRIPTION

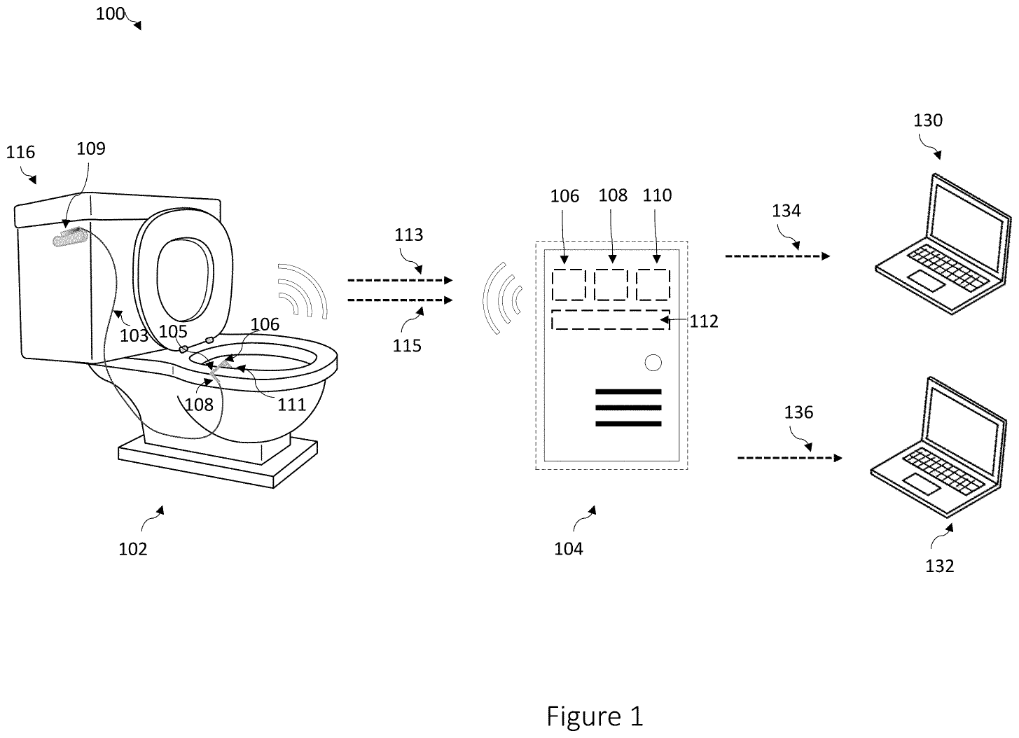

FIG. 1 illustrates a system 100 including a urinalysis device 102 that is mounted on a toilet 116 and in electronic communication with a server 104. The server 104 is in communication with a patient's computer 130 and a physician's computer 132. The urinalysis device 102 can be used to analyze urine of a patient (e.g., a patient suffering from kidney disease, a dialysis patient, etc.) and to transmit urine data 113 and patient identification data 115 to the server 104. The urine and patient identification data can be processed by the server 104 and then made accessible to the patient and/or his or her clinician for analyzing the dietary, medication, and/or blood treatment needs of the patient. For example, the processed data 134, 136 can be transmitted to a patient's computer 130 and to a physician's computer 132, as depicted in FIG. 1. This information may be used by the clinician and the patient to help the patient adhere to a recommended treatment plan. For example, the processed data can be used by the clinician and/or the patient to assess how the patient's diet, medication, and/or blood treatments are impacting the patient's health. The system 100 will be described below as being used for dialysis patients, but it should be understood that the system 100 can be used to assist any of various other types of patients that can benefit from adherence to a recommended diet, medication regimen, and/or medical treatment regimen.

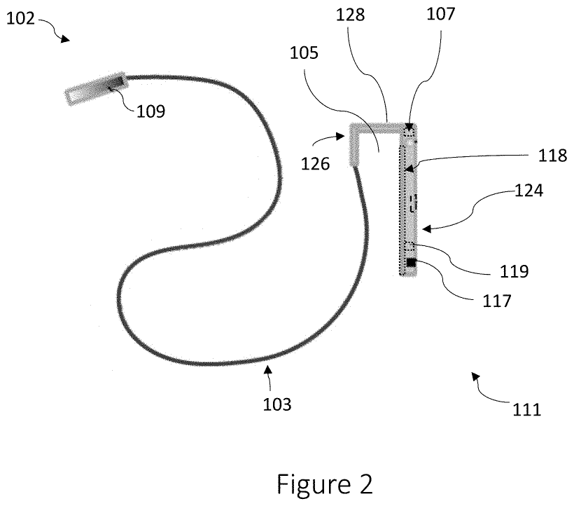

FIG. 2 shows a more detailed view of the urinalysis device 102 in an unmounted state. Referring to FIGS. 1 and 2, the urinalysis device 102 has a body 111 that is U-shaped (or hook shaped) and sized to receive the rim of the toilet 116 in an interior cavity 105. The body 111 has a first arm 124 that is longer than a second arm 126. The first arm 124 and the second arm 126 are connected by a bar 128. The first arm 124 houses a biomarker sensor 117, a water level sensor 118, a battery 120, a status light 122, and a signal transmitter 107. The urinalysis device 102 is configured such that a portion of the first arm 124 including the biomarker sensor 117 and the water level sensor 118 extends into the water in the toilet 116, as shown in FIG. 1.

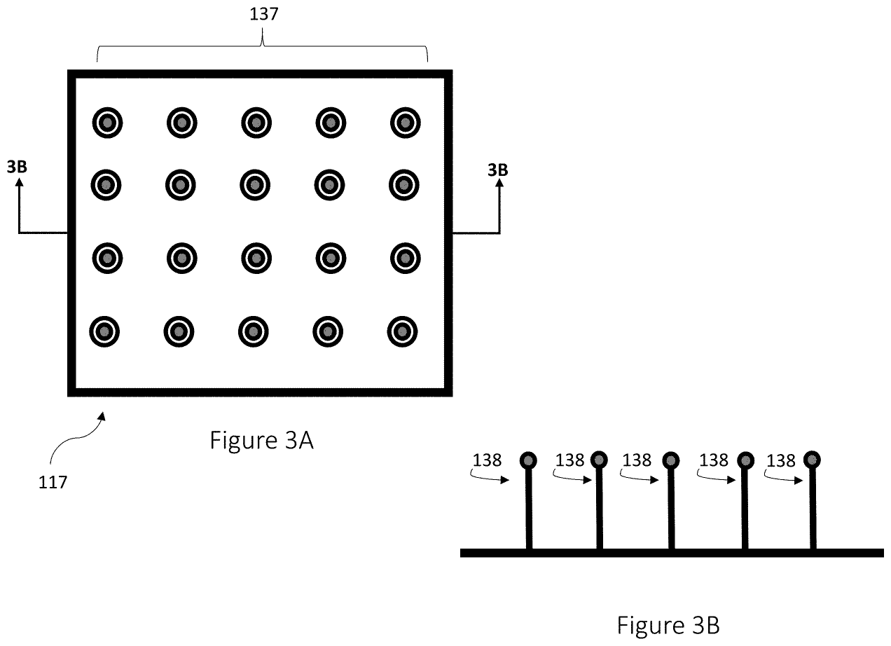

The biomarker sensor 117, which is schematically illustrated in FIGS. 3A and 3B, contains a biomarker array 137 that can detect at least one of: specific gravity, pH, uric acid, glucose, fat, fiber, proteins indicating renal infections, and molecules such as Nitrites, Sodium, Potassium, Calcium, and Phosphates, in a urine sample (e.g., urine in the toilet 116 shown in FIG. 1). Raw urine data 113 is generated by the biomarker sensor 117 of the urinalysis device 102 when the biomarker sensor 117 tests a sample of urine. That raw urine data can be transmitted to the server 104 (shown in FIG. 1) where it is processed.

As shown in FIG. 3A, the biomarker sensor 117 has arrays 137 with nanoscale resolution and active regions on the order of 1 micron. The size of the individual sensors 138, shown in FIG. 3B, within the array allows for specific screening of a variety of desired markers. For example, peptides can be exposed to a receptor and probes that bind to the receptor can then be identified. Some urinalysis devices have an array of biomarkers that each detect one or more properties of the urine.

Referring again to FIGS. 1 and 2, raw urine data generated by the biomarker sensor 117 is carried to the signal transmitter 107 through the arm 124. For example, an electrical wire or conduit can be positioned within the arm 124 to carry the data from the sensor 117 to the transmitter 107.

Still referring to FIGS. 1 and 2, a water level sensor 118 is used to detect the amount of water in the toilet 116, and a temperature sensor 119 is used to detect a temperature of the water in the toilet 116. The biomarker sensor 117 can be used to detect the composition of the toilet water (e.g., the concentration of certain components in the water that are to be later tested in a urine sample) before urine is introduced into the water. Based on known effects of water temperature and water volume on the urine components to be detected by the urinalysis device 102, the urinalysis device 102 can be calibrated based on the measured water temperature and composition before the introduction of the urine.

In some implementations, the urinalysis device is calibrated by taking the combined measurements of the water temperature, water volume or level, specific gravity, pH, uric acid levels, glucose levels, fat levels, proteins levels, fiber levels, nitrite levels, sodium levels, potassium levels, calcium levels, and phosphate levels in the water. These measurements can be taken at regular intervals, such as every 30 seconds, 1 minute, 5 minutes, 30 minutes, or 1 hour. This calibration data is used as the baseline or “zero measurement” for the subsequent sample measurements. When urine is added, concentrations will change.

The water level sensor 118 can be any of various type of suitable level sensors, including electrodes, mechanical float switches, optical sensors, capacitance sensors, and ultrasonic sensors. The temperature sensor 119 can be any of various suitable sensors capable of detecting water temperature.

A finger print reader 109, as shown in FIGS. 1 and 2, is connected to the body 111 by a wire 103. The fingerprint reader 109 sits on the toilet lever. Raw patient data 115 is generated by the finger print reader 109 when the finger print reader 109 scans a finger print of the patient when the patient presses the handle to flush the toilet. The raw patient data 115 can be carried to electronics in the body 111 via the wire 103.

A signal transmitter 107 is attached to the arm 124 of the body 111. The signal transmitter 107 can be any of various commercially available transmitters capable of being electronically connected to the wire 103, the biomarker sensor 117, and the water sensor 118. The signal transmitter 107 can be used to transmit the raw urine data 113 and the raw patient data 115 to the server 104, as schematically illustrated in FIG. 1.

A status light 122 is used to indicate an action or alert. The status light 122 can, for example, be configured to blink green to indicate that the sensor 117 is measuring, to be solidly green to indicate that the device 102 is calibrated, to blink red to indicate that raw urine and patient data 113, 115 is being transmitted, and to be solidly red to indicate that the transmission of the raw urine and patient data 113, 115 is complete.

Referring again to FIG. 1, the server 104 includes a signal receiver 106, a processor 108, a controller 110, and a memory 112. The signal receiver 106 in the server 104 receives the raw urine data 113 from the signal transmitter 107 of the urinalysis device 102. The processor 108 in the server 104 processes the raw urine data 113 to determine levels and concentrations of components in the urine of the patient which are indicative of patient diet or health. The processor 108 can, for example, detect or determine one or more of the following properties or characteristics of the urine: specific gravity, pH, uric acid levels, glucose levels, fat levels, protein levels that indicate renal infections, fiber levels, and molecules such as nitrites, sodium, potassium, calcium, and phosphates. To access the server 140, the medical professional (clinician) downloads a program on the medical professional's computer 132 that allows viewing and editing access to the server 104, such that a m may view or edit data in the server 104. In the initial setup of the program, the clinician selects one or more software scripts to be a set of default software scripts. The software scripts are executed by the processor 108 to generate processed data from the raw urine data 113. Each of the software scripts may focus on at least one of the properties detected by the biomarker sensor 117 and include outputs in the form of graphs or reports. Software scripts other than the set of default software scripts may be run after the set of default software scripts.

The signal receiver 106 also receives raw patient identification (ID) data 115 from the signal transmitter 107 of the urinalysis device 102. The processor 108, the controller 110, and the memory 112 of the server 104 compare and tag the raw patient ID data 115 with a patient ID stored in the memory 112. The memory 112 includes a data set of predetermined patient ID data. The controller 110 and the processor 108 compare the predetermined patient ID data to the raw patient ID data 115. When the predetermined patient ID data in the memory 112 matches the raw patient ID data 115, the raw patient ID data 115 is tagged with the corresponding predetermined ID data (matched patient ID). When the predetermined ID data in the memory 112 does not match the raw patient ID data 115, the raw patient ID data 115 is tagged as an unknown person (unmatched patient ID).

The most recent raw urine data 113 received by the server 104 is then tagged with the patient ID now associated with the raw patient ID data 115. At this step, the raw urine data 113 and the raw patient ID data 115 are tagged by the same patient ID. The data 113, 115 is then saved in the memory 112 of the server 104 under a folder associated with the patient ID.

When the patient ID is an unmatched patient ID, the data 113, 115 is saved with other data 113, 115 tagged as an unknown patient. In some cases, the data 113, 115 tagged as an unknown person is automatically deleted after a predetermined period of time (e.g., one hour, one day, one week, one month, one year, etc.), assuming no valid patient ID associated with that data is saved in the server 104 before that time period expires. In other cases, the data 113, 115 determined to be associated with an unknown person is automatically deleted immediately upon making that determination.

The processor 108 processes the raw urine data 113 and inputs the most recent levels of components (the processed data) in the urine into a folder associated with the patient ID. This occurs for both matched patient IDs and unmatched patient IDs. To process the raw urine data 113, the processor 108 runs the set of default software scripts, analyzes the raw urine data 113, and identifies key markers or patterns in the raw urine data. The set of default software scripts are initially chosen by the clinician during the program setup in the medical facility. Once the processor 108 analyzes the raw urine data 113 using the set of default scripts, the processor 108 outputs processed urine data.

The memory 112 of the server 104 saves the processed data under the folder associated with the patient ID. If the patient ID is a matched patient ID, the controller generates a packet of data that includes processed urine data associated with a patient ID data. The controller 110 of the server 104 generates a report from the packet of data related to the levels of components measured in the urine. These reports are transmitted to the patient's computer 130 and/or to the medical professional's computer 132. These reports can be viewed by the patient, physician, and/or nutritionist and used to adjust the patient's treatment plan (e.g., the patient's recommended diet, prescribed medication(s), and/or recommended blood treatment parameters).

As noted above, if the patient ID is an unmatched patient ID (unknown patient), the processed urine data, the raw urine data, and the raw patient ID data is saved under the finder associated with unknown patients. The controller 110 deletes the data accompanied with an unmatched patient ID after a given period time. Prior to the deletion of this data, a user with access to the server 104 may review the folder containing the unmatched patient IDs and the associated data. If the user determines that the urine data belongs to a known patient, the user can manually retag the data and can move the data to the proper folder of a known patient ID.

A method of using the system 100 illustrated in FIG. 1 will now be described. As a first step, a patient would go to his or her physician and would be diagnosed with kidney disease or another ailment that would benefit from use of the system 100. After receiving this diagnosis, the patient would obtain the urinalysis device 102. The urinalysis device 102 could, for example, be supplied to the patient by the physician or the patient could go purchase the urinalysis device. The patient would then install the urinalysis device 102 in a toilet in the home of the patient so that biological markers could be tracked by the patient and his or her physician on a regular basis.

After installing the urinalysis device 102, the urinalysis device 102 is automatically calibrated on a regular basis to generate baseline data. The urinalysis device 102 can, for example, be calibrated every 30 seconds, one minute, 30 minutes, one hour, etc. As discussed above, the urinalysis device 102 can be calibrated based on the detected level and composition of the water in the toilet 116 prior to introducing urine into the toilet 116. More specifically, the biomarker sensor 117 detects the initial levels of pH and other water properties to create a base line. The water level sensor 118 and the temperature sensor 119 detect the amount of water in the toilet 116 and the temperature of that water. After the calibration process is complete, the status light 122 emits a solid green light to indicate that the device is calibrated and ready to test a urine sample.

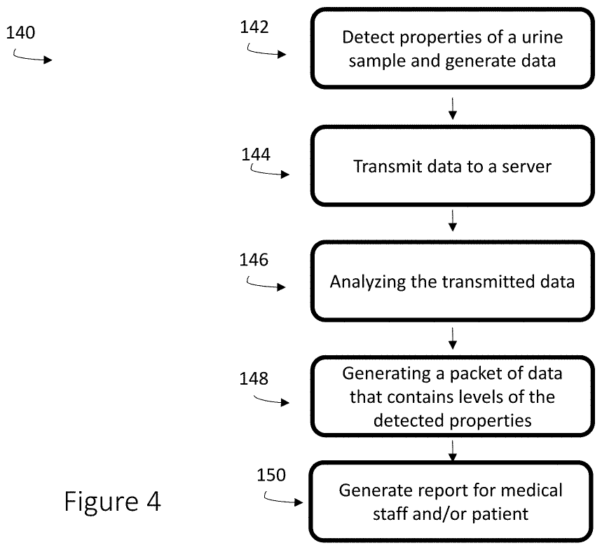

FIG. 4 illustrates a portion of the method of using the system 100. Referring to FIG. 4, after the patient has urinated in the toilet 116, the biomarker sensor 117 delays measurement for a period of time (e.g., approximately 10 seconds, 15 seconds, 20 seconds, etc.) so that the urine becomes uniformly diluted by the toilet water before the measurement is taken. Then the sensor 117 measures and detects the properties of the urine 142 and generates raw urine data 113. During this time, the status light 122 blinks green to indicate that the sensor 117 is taking measurements. The patient will not flush the toilet until the status light 122 stops blinking, indicating that the measurement is complete.

Once the measurement is complete, the user places a finger on the fingerprint reader 109 (e.g., to flush the toilet). The finger print reader 109 scans the finger print and generates raw patient ID data. The raw patient ID data 115 is transmitted 144 to the signal receiver 106 of the server 104. The status 122 light blinks red to indicate that the data is being transmitted and is solidly red once the data has been successfully transmitted. Advantageously, the finger print reader 109 can automatically scan the patient's finger print as the patient flushes the toilet such that no additional steps beyond those typically involved with using a toilet are required.

Flushing of the toilet 116 cleans the biomarker sensor 117. Once fresh water fills the toilet bowl, the urinalysis device 102 recalibrates so it will be ready for a subsequent use.

The signal transmitter 107 then transmits the raw urine data 113 and the raw patient ID data 115 to the signal receiver 106 of the server 104 (144). Transmission of the data 113, 115 to the server 104 can be triggered by the finger print reader 109 detecting a finger print. Alternatively, transmission the data 113, 115 can be triggered in other ways, such as by the water level sensor 118 detecting a significant drop in the water level indicating that the toilet 116 has been flushed.

The processor 108, the controller 110, and the memory 112 of the server 104 are then used to analyze the data 113, 115 (146). The processor 108, the controller 110, and the memory 112 of the server 104 process, compare, and tag the raw patient ID data 115 with a patient ID stored in the memory 112. The memory 112 includes a data set of predetermined patient ID data that are each associated with a patient ID. The controller 110 and the processor 108 compare the predetermined patient ID data to the raw patient ID data 115. When the predetermined patient ID data in the memory 112 matches the raw ID data 115, the raw patient ID data 115 is tagged with the patient ID associated with the predetermined patient ID data. The most recent raw urine data 113 received by the server 104 is also tagged with the patient ID associated with the predetermined patient ID data to associate the urine data with the known patient.

The processor 108 in the server 104 then processes the raw urine data 113 to determine levels and concentrations of components in the urine of the patient which are indicative of patient diet and/or health 146. The raw urine data 113 is processed using the set of default software scripts chosen during the initial setup of the program in the medical facility. The processor 108 in the server 104 generates a packet of processed urine data 148 that contains levels and concentrations of the detected properties in the urine sample. This packet of data is analyzed and a report is generated 150 based on the packet of urine data. The report can identify properties of the urine that are outside a predetermined range. The report can also estimate a diet of the patient based on the packet of data and can thus allow the patient and his or her physician to determine how closely the patient has been adhering to a recommended diet. In some cases, the report includes suggested adjustments to the patient's treatment plan. The report can, for example, recommend adjustments to the patient's diet, recommend adjustments to the patient's medication(s) or medication dosage(s), or recommend adjustments to blood treatment parameters for an upcoming blood treatment.

The report is transmitted to a medical professional's computer 132 and/or to the patient's computer 130. At a patient's next appointment, the medical professional may review the report and adjust the treatment plan as needed. For example, if uric acid levels are higher than expected, a medical professional may adjust medication dosages. Or if potassium levels are low, a medical professional may recommend increasing potassium-rich foods in the patient's diet like bananas or prescribe potassium supplements.

For patients who are receiving blood treatments (e.g., dialysis treatments), the report can be used by the medical professional to determine an appropriate blood treatment (e.g., dialysis) prescription. For example, if the patient is determined to be overly hydrated, which can be determined based on the detected specific gravity and/or ion concentration levels, the blood treatment prescription may be adjusted to increase the ultrafiltration volume. The report can also suggest a specific concentration of dialysate to be used for the patient based on the detected properties of the patient's urine (e.g., based on molecular levels of nitrate, sodium, potassium, calcium, and/or phosphate in the patient's urine).

For patients who are not yet receiving blood treatments (e.g., dialysis treatments), the report can be used by the medical professional to determine when blood treatments may become necessary for those patients.

Thus, the processed urine data can be used by the patient and his or her physician to improve a number of different aspects of the patient's treatment plan and can be used for patients in various different stages of kidney disease.

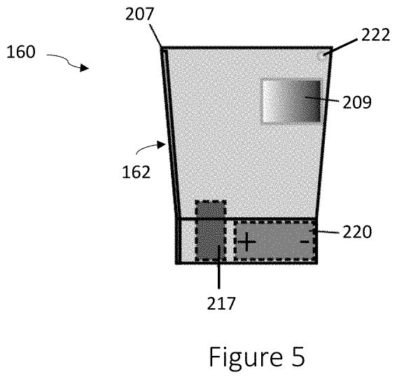

FIG. 5 shows another urinalysis device 160 for generating raw urine data and transmitting raw urine data. Like the urinalysis device 102, the data obtained from the urinalysis device 160 can help a patient and medical professional adhere to, evaluate, and/or modify a treatment plan. The urinalysis device 160 has a cup shaped body 162. A battery 220, a biomarker sensor 217, a status light 222, and a signal transmitter 207 are disposed on or in the body 162. A fingerprint reader 209 is attached to the outside of the body 162 so that the fingerprint reader 209 is accessible to the user. The urinalysis device 160 operates similarly to the urinalysis device 102 with some key differences. Like the urinalysis device 102, the urinalysis device 160 is calibrated before use. The baseline, however, is when the device 160 is empty. The biomarker sensor 217 in the cup shaped body 162 calibrates by washing or rinsing the device 160 (rather than through flushing). There is no water sensor. When a patient urinates into the cup, the biomarker sensor 217 detects the same parameters as the biomarker sensor 117. The biomarker sensor 217 may differ in sensitivity relative to the biomarker sensor 117 in the urinalysis device 102 since the urine sample is not diluted in the urinalysis device 160. The signal transmitter 207 connects to the biomarker sensor 217 to transmit the raw urine data to the server 104. The signal transmitter 207 also connects to the fingerprint reader 209 to transmit the raw patient data 115 to the server 104. The status light 222 is similar to the status light 122 in the urinalysis device 102. The status light 222 is used to indicate an action or alert. The status light 222 blinks red to indicate data is being transmitted, blinks green to indicate that the biomarker sensor 217 is measuring, is solidly green to indicate that the device 160 is calibrated, and is solidly red to indicate that data 113, 115 is sent. The cup may be reused by simply pouring out a previous sample and rinsing the device 160 with water.

While urinalysis devices 102, 160 with fingerprint readers have been previously described, the urinalysis device may instead include a radio-frequency identification (RFID) to confirm patient identification. RFID is a wireless technology that uses radio waves to transfer data from a RFID tag, an electronic tag attached to an object, for the purpose of identifying the object. For example, a RFID tag might be attached to a patient's phone or watch and uniquely identify that patient. When the patient uses the urinalysis devices 102, 160, the RFID tag can be identified and a patient's identity tagged to the urinalysis data detected. Other biometric sensors to confirm the patient ID, such as iris/retina scans, heat signatures, or voice recognition can alternatively or additionally be used.

In some implementations, a patient ID code is entered into the system by the patient to associate the tested urine with that patient. For example, the urinalysis device may be connected to a keypad that is mounted to the toilet. The system provides the patient with a unique patient ID code, for example “012345”, that is entered directly before providing a sample or directly after providing a sample. Alternatively the patient may program a patient ID code when setting up the device for the first time, either on the device or on a website. The patient ID code is linked to a patient profile in a remote server. The patient profile includes urinalysis data taken by the urinalysis device, or any other urinalysis device compatible or connected to the remote server. Other information can also be stored in the patient profile, for example, manually entered urinalysis data, blood treatment prescriptions, treatment history, and other data relevant to blood treatment. Once the urinalysis device has detected and measured the sample, raw urine data is generated. The urinalysis device transmits raw urine data to the remote server if a patient ID code has been entered within a predetermined amount of time of a detected sample, for example within 3 minutes of detecting and measuring a sample. Otherwise the raw urine data is deleted. The transmitted raw urine data is tagged with the manually entered patient ID code. The remote server receives the tagged raw urine ID and sorts the raw urine ID into the patient profile associated with the patient ID code.

Some urinalysis devices may confirm the patient ID prior to send the raw urine data to the server. In such a case, the patient enters the patient ID code either before or after the sample the provided. The urinalysis device transmits the patient ID code to the server, and the server retrieves the patient name associated with the entered patient ID code. The name of the patient is then transmitted from the server to the urinalysis device and the urinalysis device displays the name on a user interface of the keypad. Once the user confirms that the displayed name is correct, the raw urine data is transmitted to the server.

While signal transmitters in direct wireless communication with a server have been described, the urinalysis device may instead include a low range signal transmitter, for example Bluetooth, that connects to a second device such as a local network, smart phone, tablet, laptop, or computer. The low range signal transmitter connects to the second device, and the second device wirelessly connects to the server. In this configuration, the low range signal transmitter extends the life of the battery of the urinalysis device. Some urinalysis devices have both a low range transmitter connected to a second device and a long range signal transmitter wirelessly connected to the server. The report generated from the received raw urine data 113 can then be sent to either or both the phone and the server. The signal transmitters may also connect to a router first and then by wire to a server.

While the server 104 has been described as being in communication with the patient's computer and the physician's computer, in some implementations, the server is in communication with a blood treatment machine to be used to perform a blood treatment on the patient. In such implementations, a packet of processed data related to the levels of detected properties in the patient's urine is be sent to a blood treatment machine. The blood treatment parameters to be programmed into the blood treatment machine for the patient's blood treatment can be adjusted based on the packet of processed data. In some cases, the data sent to the blood treatment machine includes an entire blood treatment prescription that the clinician can accept in one easy step to carry out the treatment using that prescription. Alternatively, the clinician may review the data and then input the desired blood treatment parameters.

Additionally, to avoid transmitting erroneous patient data, the urinalysis device may transmit the raw urine data 113 only when the patient's ID is detected by the urinalysis device (e.g., by from an NFC/RFID tag carried by the patient, the patient's fingerprint, or any of the various other patient identification mechanisms described herein) and confirmed as the known patient. In such a case, the urinalysis device would include a controller and a memory that contains the predetermined patient ID data. Once the patient's ID was confirmed, the urinalysis device uses the controller of the urinalysis device to tag the raw urine data with the matched patient ID. The signal transmitter then transmits the tagged, raw urine data to the server for processing and analysis. In this configuration, the urinalysis device may discard the urine data of any unmatched patient ID.

While the status lights have been described as using specific light patterns to indicate different states of the urinalysis devices, the status lights can alternatively or additionally emit different colors to indicate different actions performed by the urinalysis device. For example, the status light can be red when it is calibrating and cannot receive a urine sample, green when it is ready to receive a urine sample, and yellow when the raw urine sample is being transmitted by the signal transmitter of the urinalysis device. The status light could also have different blinking patterns to indicate the status of the urinalysis device.

In some implementations, the calibration step and be prompted by a controller of the urinalysis device. The controller can, for example, determine when the toilet is flushed using the water level sensor. When the toilet is flushed, the water level of the toilet rises and falls. The controller recognizes the water level movement using the water level sensor and prompts calibration. The controller may also prompt the biomarker sensor to measure the sample when the water level sensor detects an increase in water level (due to the addition of a urine sample).

While programs that allow access to the server 104 have been described as requiring the user to select a set of default software scripts as a part of the initial setup of the program, some programs may instead require the clinician to choose software scripts during the initial setup of each urinalysis device, so that each patient has a customized set of one or more software scripts analyzing his/her raw urine data. For example, the clinician may assign one patient a software script that analyzes the proteins in the urine detected by the biomarker sensor while the clinician assigns another patient a software script that analyzes the glucose levels and pH detected by the biomarker sensor. In some programs, the program requires the user to select a set of default software scripts during the initial setup of the program and requests that the user select a customized set of software scripts when the urinalysis device is initially setup. The default set of software scripts and/or the customized set of software scripts may be changed using the program.

While the processing device that connects to the urinalysis device has been described as a server, the processing device may be any device with adequate processing capability, such as a mobile phone, a tablet, a computer, etc. asked

While the finger print reader 109 has been described as being positioned on the flushing lever of the toilet 116, it could alternatively be positioned at any of various other locations in proximity to the toilet 116.

In some systems, the structure of the urinalysis devices may vary. Instead of a hook-like structure, the urinalysis device may be clipped onto the toilet or mounted in a different way so that part of the device remains in contact with the toilet water. The urinalysis device may also be in a stick-shape instead and or disposable.

While the server has been described as being in communication with a patient's computer 130 and a physician's computer 132, the server can alternatively or additionally be in communication with any of various other electronic devices of the patent and the clinician, including mobile phones, tablets, etc.

While the server 104 has been described as transmitting a report to a patient's computer and/or to a medical professional's computer, the data transmitted need not be in the form of a formal report. Rather, the data can be in any form that is usable by the patient and/or the medical professional.

While the urinalysis device 102 has been described as being installed in a toilet at the patient's home, it could alternatively be installed in any toilet to be used by the patient. In some cases, for example, the urinalysis device 102 is installed in a toilet at a dialysis clinic. In such cases, the patient may be asked to urinate in that toilet immediately prior to his or her treatment to get the most up to date data prior to the treatment. That data can then be used to modify the planned treatment if desired.

Other embodiments are within the scope of the following claims.

1. A method comprising:
sensing properties of a urine sample of a patient using a biomarker sensor to generate raw urine data;
transmitting the raw urine data to a processor,
using the processor to generate processed urine data from the raw urine data, and
transmitting the processed urine data to a computing device.

2. The method according to claim 1, further comprising suggesting one or more adjustments to a treatment plan based on the processed urine data.

3. The method according to claim 2, wherein the treatment plan is at least one of a nutrition plan, a medication prescription, and a blood treatment prescription.

4. The method according to claim 1, wherein the processed urine data comprises a packet of urine data that contains levels and concentrations of the sensed properties in the urine sample.

5. The method according to claim 1, further comprising sensing a patient identification, generating patient identification data, and transmitting the patient identification data to the processor.

6. The method according to claim 5, further comprising comparing the transmitted patient identification data to predetermined patient identification data and determining a patient identification.

7. The method according to claim 6, further comprising linking the processed urine data to the patient identification.

8. The method according to claim 5, further comprising linking the transmitted raw urine data to the transmitted patient identification data.

9. The method according to claim 5, wherein sensing the patient identification comprises sensing a fingerprint using a fingerprint reader.

10. The method according to claim 5, wherein sensing the patient identification comprises sensing an RFID tag using an RFID detector.

11. The method according to claim 1, wherein transmitting the raw urine data to the processor comprises:
transmitting the raw urine data using a signal transmitter of a urinalysis device,
receiving the raw urine data using a signal receiver of an intermediate device, and
transmitting the raw urine data to the processor using a signal transmitter of the intermediate device.

12. The method according to claim 11, wherein the intermediate device is a mobile computing device.

13. The method according to claim 12, wherein the mobile computing device is a mobile phone or a tablet.

14. The method according to claim 1, further comprising using the processor to adjust parameters of a blood treatment prescription based on the processed urine data.

15. The method according to claim 14, further comprising transmitting the blood treatment prescription with the adjusted parameters to a blood treatment machine.

16. The method according to claim 14, wherein the processed urine data comprises at least one of specific gravity, pH, uric acid levels, glucose levels, fat levels, proteins levels, fiber levels, nitrite levels, sodium levels, potassium levels, calcium levels, and phosphate levels.

17. The method according to claim 1, further comprising generating a report based on the processed urine data.

18. The method according to claim 17, wherein the report identifies properties of the urine sample that are outside a predetermined range.

19. The method according to claim 17, further comprising determining a diet of a patient based on the processed urine data, wherein the report identifies the diet of the patient.

20. The method according to claim 17, further comprising transmitting the report to a medical professional.

21. The method according to claim 17, further comprising transmitting the report to a patient.

22. A urinalysis device comprising:
a body configured to be connected to a toilet,
a biomarker sensor attached to a portion of the body configured to be submerged in water in the toilet when the body is connected to the toilet, the biomarker sensor configured to detect properties of urine and to generate raw urine data comprising the detected properties of the urine, and
a signal transmitter configured to transmit the raw urine data to a processing device.

23.-39. (canceled)

30. A system comprising:
a urinalysis device comprising:
a body configured to be connected to a toilet,
a biomarker sensor attached to a portion of the body configured to be submerged in water in the toilet when the body is connected to the toilet, the biomarker sensor configured to detect properties of urine and to generate raw urine data comprising the detected properties of the urine, and
a signal transmitter configured to transmit the raw urine data; and
a processing device comprising:
a signal receiver configured to receive the raw urine data from the signal transmitter of the urinalysis device,
a processor configured to process the raw urine data and generate processed urine data from the raw urine data, and
a memory for storing the processed urine data.

31.-37. (canceled)
